(19) United States
(12) Patent Application Publication
(10) Pub. No.: US 2018/0036341 A1
(43) Pub. Date: Feb. 8, 2018

(54) METHOD OF USING CARBONIZED MATERIAL TO INHIBIT BACTERIA

(71) Applicant: LINKWIN TECHNOLOGY CO., LTD., Taichung City (TW)

(72) Inventor: YI-CHING CHENG, TAICHUNG CITY (TW)

(73) Assignee: LINKWIN TECHNOLOGY CO., LTD., TAICHUNG CITY (TW)

(21) Appl. No.: 15/226,587

(22) Filed: Aug. 2, 2016

(51) Int. Cl. A61K 33/40; A61F 13/00; C25B 1/30

(52) U.S. Cl. CPC A61K 33/40; C25B 1/30; A61F 13/00063; A61F 13/00017

(57) ABSTRACT

A method of using a carbonized material to inhibit bacterial growth is provided, wherein the method includes the steps below. First, prove the carbonized material which includes an electrically conductive carbon-containing body, wherein the carbon-containing body is selected from the group consisting of carbon fiber fabric, graphene, and graphene oxide. Next, expose the carbonized material to a wet environment. Finally, provide a direct current to the carbon-containing body. Whereby, after the carbon-containing body is electrically energized by the direct current, hydrogen peroxide is synthesized form oxygen in the wet environment to inhibit bacterial growth.

### BACKGROUND OF THE INVENTION

### 1. Technical Field

The present invention relates generally to a method of using a carbonized material to inhibit bacteria, and more particularly to a method of using a material with an electrically conductive carbon-containing body to inhibit the growth of bacteria on wound.

### 2. Description of Related Art

People inadvertently suffer injuries (e.g., cutting injuries, abrasions, and burns) in daily lives. If the wound is not properly treated, the prognosis is usually poor, and the wound may be even infected. Especially, controlling of extensive burns is extremely difficult among the common injuries. Skin is a protective barrier over the body's surface, and once skin is seriously injured, it would cause the risk of dehydration and hypothermia. Moreover, if the wound is not well treated, the patient may have a severe infection a few days later, potentially leading to respiratory failure, multiple organ failures, or even death. In short, it is challenging for healthcare institutions to not only sustain burn patients, but also reconstruct the protective barrier (i.e., the skin) of patients.

If the depth of the burn extends deep into the reticular dermis, burn patients have to undergo skin autografting to prevent transplant rejection. However, skin substitutes (e.g., artificial skin or wound dressing) are needed for those patients who don't have enough healthy skin remained, or for extra protection over the treated wound. Currently, artificial skins with algin and silicone gauze are commonly used, wherein the former can keep the wound moist, and the latter is non-adhesive to the wound, and thus, these two kinds of artificial skins could both facilitate the healing.

However, the conventional skin substitutes mentioned above are all unable to effectively inhibit the growth of bacteria, and therefore, have to be replaced after being applied for a period of time. Consequently, it would consume a large amount of medical materials. Furthermore, replacing wound dressing frequently would be quite painful for burn patients. Therefore, seeking a better method for healing burn patients is a pressing issue.

### BRIEF SUMMARY OF THE INVENTION

In view of the above, the primary objective of the present invention is to provide a method of using a carbonized material to cover a wound for inhibiting bacteria, which has a continuously and significantly antibiotic effect.

The present invention provides a method of using a carbonized material to inhibit bacterial, including providing the carbonized material which includes an electrically conductive carbon-containing body, exposing the carbonized material to a wet environment, and providing a direct current to the carbon-containing body, wherein the carbon-containing body is selected from the group consisting of carbon fiber fabric, graphene, and graphene oxide. Whereby, after the carbon-containing body is electrically energized by the direct current, hydrogen peroxide is synthesized form oxygen in the wet environment to inhibit bacterial growth.

Whereby, a small amount of hydrogen peroxide is synthesized by an electrochemical action to inhibit the growth of bacteria on the wound.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a schematic diagram of a first embodiment of the present invention, showing the carbonized material;

FIG. 2 is a partial schematic diagram of the carbonized material in FIG. 1, showing carbon fiber fabric includes a plurality of carbon fibers arranged in a crisscross pattern;

FIG. 3 is a flow chart of the method of using the carbonized material in FIG. 1; and

FIG. 4 is a schematic diagram of using the carbonized material in FIG. 1.

### DETAILED DESCRIPTION OF THE INVENTION

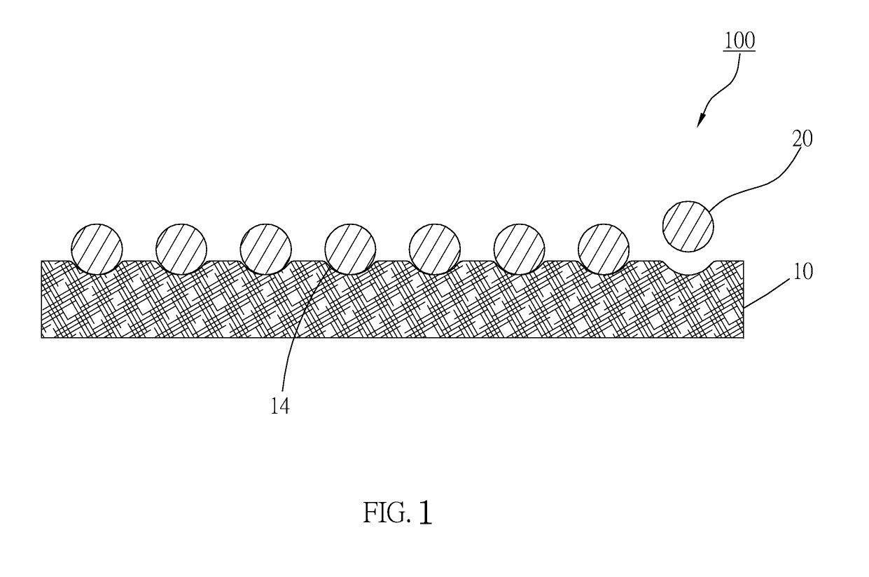

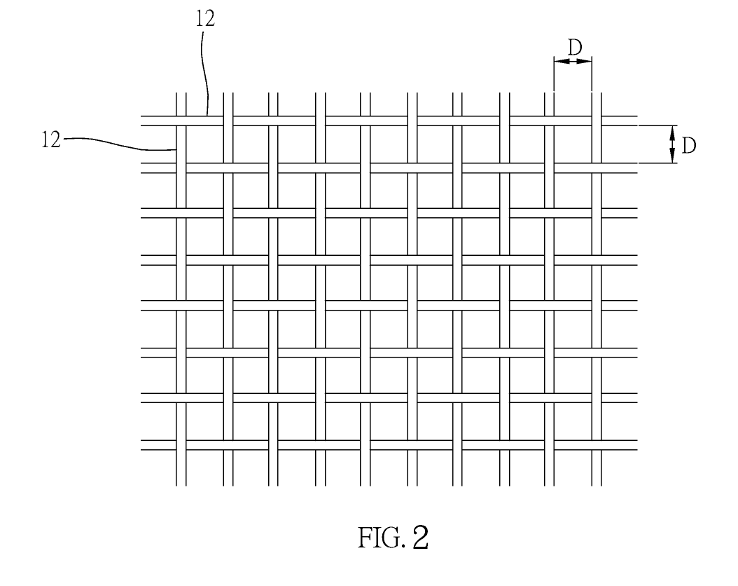

As shown in FIG. 1, the carbonized material 100 includes a carbon-containing body 10 and a plurality of silver particles 20 adhered to the carbon-containing body 10. The carbon-containing body 10 is electrically conductive, and is at least one of carbon fiber fabric (CF), graphene, and graphene oxide (GO). The carbon fiber fabric has excellent adsorption characteristic and moisture retention. As illustrated in FIG. 2, the carbon fiber fabric includes a plurality of carbon fibers 12, wherein a distance D between two adjacent carbon fibers 12 among the plurality of carbon fibers is between 0.2 nm and 0.25 nm; in the embodiment, the distance D is 0.23 nm. Practically, the carbon-containing body 10 can be the porous fiber fabric (activated carbon fiber fabric) disclosed in Taiwan patent 1334891 filed by the applicant of this invention. Moreover, the carbon-containing body 10 includes a multilayer structure which has a plurality of layers stacked; each of the layers is graphene or graphene oxide.

A ratio by weight of the silver particles 20 and the carbon-containing body 10 is between 0.5 to 5; in such range, the interplanar distance of the (111) plane of Ag in the carbonized material 100 is 0.23 nm, which is prone to attach to the carbon-containing body 10. Additionally, the best ratio by weight of the silver particles 20 and the carbon-containing body 10 is 5, the preferred ratio is 1, and the next is 0.5. The silver particles 20 and the carbon-containing body 10 are mixed in liquid. In the embodiment, the ratio by weight of the silver particles 20 and the carbon-containing body 10 is 2. The carbonized material 100 can effectively adsorb wound exudate, blood, and bacteria when covering a wound; moreover, it can keep the wound wet.

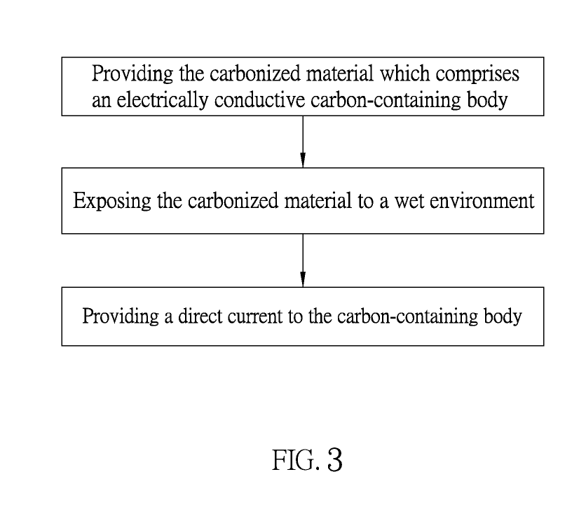

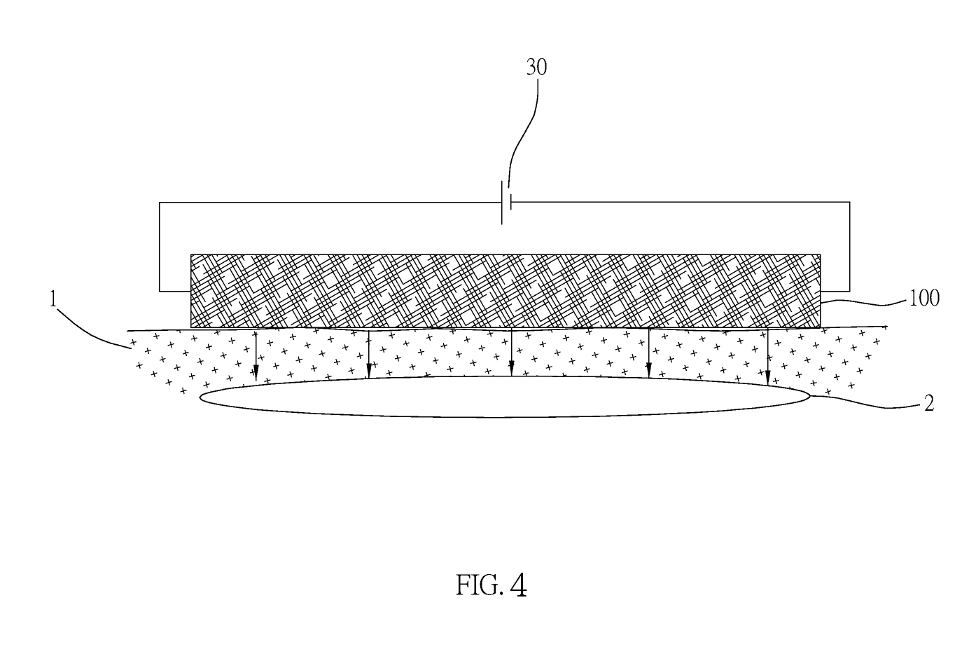

FIG. 3 to FIG. 4 shows the method of using the carbonized material 100 in inhibiting bacteria. First, expose the carbonized material 100 to a wet environment 1 which has oxygen. In practical, the wet environment 1 is a wound of an organism. In other words, the carbonized material 100 directly covers the wound. Next, provide a direct current to the carbon-containing body 10, wherein the direct current is provided by a power supply 30 which provides a voltage of 1.75 N (1.75 multiples) volt, wherein N is a positive integer, and further satisfies:

1≦N≦8;

in the embodiment, N=2, and the power supply 30 is a battery providing a voltage of 3.5 volt.

After the electrically conductive carbon-containing body 10 is electrically energized, low concentration of hydrogen peroxide 2 is synthesized form oxygen in the wet environment 1 by an electrochemical action, and is released to the surface of the wound to inhibit the growth of bacteria on the wound. If the voltage provided form the power supply 30 is too high, the concentration of the hydrogen peroxide 2 would be too high, which may damage normal tissues of the organism. In contrast, if the voltage provided form the power supply 30 is too low, the concentration of the hydrogen peroxide 2 would be too low to effectively inhibit the growth of bacteria.

In conclusion, after the carbon-containing body 10 electrically energized, a small amount of hydrogen peroxide 2 would be released and subsequently inhibit the proliferation of bacteria on the wound, which preliminary inhibit bacteria. Yet a few remained bacteria growing with hydrogen peroxide 2 would be attracted by the carbonized material 100, and further be impaled by the silver particles 20 thereof, which leads to the death of the bacteria. In this sense, the carbonized material 100 can significantly reduce the number of the bacteria on the wound by the abovementioned dual antibacterial effect, which effectively prevents wound infection. In addition, the multilayer structure in the carbon-containing body 10 consisting of graphene or graphene oxide could be barriers to bacteria, which prevents bacteria from proliferation and forming colonies on large wounds.

Additionally, moisture level in wounds is an important factor in wound healing. If a wound is not moisturizing enough, the prognosis is usually poor. With the carbon fiber fabric which has excellent moisture retention characteristic, the carbonized material 100 can effectively keep wounds wet.

With the characteristics mentioned above, the carbonized material 100 is suitable for large wounds, such as treatment for large burns. Furthermore, the power supply 30 could continuously provide electric current to the carbon-containing body 10 such that the carbonized material 100 could continuously release hydrogen peroxide 2 to the surface of the large wound, which enhances the inhibition of bacteria, and accelerates wound recovery. For burn patients, the carbonized material 100 in the invention is indeed helpful for wound healing.

It must be pointed out that the embodiments described above are only some preferred embodiments of the present invention. All equivalent methods which employ the concepts disclosed in this specification and the appended claims should fall within the scope of the present invention.

1. A method of using a carbonized material to inhibit bacterial growth, comprising the steps of:
A. providing the carbonized material which comprises an electrically conductive carbon-containing body, wherein the carbon-containing body comprises carbon fiber fabric;
B. exposing the carbonized material to a wet environment; and
C. providing a direct current to the carbon-containing body;
whereby, after the carbon-containing body is electrically energized by the direct current, hydrogen peroxide is synthesized form oxygen in the wet environment to inhibit bacterial growth.

2. The method of claim 1, wherein the carbon-containing body comprises a multilayer structure which has a plurality of layers; each of the layers is graphene or graphene oxide.

3. The method of claim 1, wherein the carbon fiber fabric comprises a plurality of carbon fibers; a distance between two adjacent carbon fibers among the plurality of carbon fibers is between 0.2 nm and 0.25 nm.

4. The method of claim 1, wherein the carbonized material further comprises a plurality of silver particles adhered to the carbon-containing body; a ratio by weight of the silver particles and the carbon-containing body is between 0.5 to 5.

5. The method of claim 1, wherein the direct current is provided by a power supply which provides a voltage of 1.75 N volt.

6. The method of claim 5, wherein N is a positive integer, and further satisfies:
1≦N≦8.

7. The method of claim 1, wherein the wet environment comprises a wound of an organism; whereby, a small amount of hydrogen peroxide is synthesized by an electrochemical action to inhibit the growth of bacteria on the wound.
